(19) United States
(12) Patent Application Publication
(10) Pub. No.: US 2021/0292714 A1
(43) Pub. Date: Sep. 23, 2021

(54) LIVER SUPPORT SYSTEM COMPRISING LIVER ORGANOIDS AND METHODS OF MAKING AND USING SAME

(71) Applicant: CHILDREN'S HOSPITAL MEDICAL CENTER, Cincinnati, OH (US)

(72) Inventors: Takanori Takebe, Cincinnati, OH (US); Jorge A. Bezerra, Cincinnati, OH (US)

(21) Appl. No.: 17/250,899

(22) PCT Filed: Sep. 27, 2019
(86) PCT No.: PCT/US19/53408

§ 371 Date: Mar. 22, 2021

Related U.S. Application Data
(60) Provisional application No. 62/737,261, filed on Sep. 27, 2018.

(51) Int. Cl. C12N 5/071; A61F 2/02

(52) U.S. Cl. CPC C12N 5/0671; A61F 2/022; C12N 2501/119; C12N 2533/90; C12N 2501/155; C12N 2501/727; C12N 2513/00; C12N 2501/16

(57) ABSTRACT

Disclosed herein are bioartificial liver systems (“BAL” or “liver assist device”) that employ liver organoids. The disclosed systems may be used to provide a therapeutic benefit to an individual in need thereof, particularly an individual having compromised liver function. The systems may include an in vivo or ex vivo component, wherein a liver function is provided to the individual using the disclosed systems.

### CROSS REFERENCE TO RELATED APPLICATIONS

This application claims priority to and benefit of U.S. Provisional Application Ser. No. 62/737,261, entitled “Liver Support System Comprising Liver Organoids and Methods of Making and Using Same,” filed Sep. 27, 2018, the contents of which are incorporated herein in its entirety and for all purposes.

### BACKGROUND

Most patients admitted to an intensive care unit in liver failure do not survive. Mortality as high as 80-90% has been reported. The only available treatment to date is hepatic transplantation. However, livers for transplantation may not be immediately available, or patients may, in certain circumstances, not qualify for transplantation.

To treat liver disease, such as acute liver failure and chronic liver diseases (such as hepatocellular carcinoma and liver cirrhosis), various approaches have been suggested, including hepatocyte transplantation, liver support systems and 3D organ printing. Currently, hepatocytes have been the most promising source of liver cells for use in extracorporeal devices to assist compromised liver function in a patient in need thereof. To date, however, the use of hepatocytes has proved to be insufficient—hepatocytes have a short lifespan when used with existing devices to assist liver function, thereby limiting the utility of such cell types. The lack of biologically-based cell systems has delayed wider clinical application of such systems, and created a need for improved methods for providing liver support systems.

While various types of liver support systems have been suggested or described, devices comprising biological components are presently lacking. The instant disclosure addresses one or more of the aforementioned needs in the art.

### BRIEF SUMMARY

Disclosed herein are bioartificial liver systems (“BAL” or “liver assist device”) that may employ liver organoids to compromised liver function in an individual in need thereof, particularly an individual with compromised liver function. The disclosed systems may be used to provide a therapeutic benefit to an individual in need thereof, particularly an individual having compromised liver function. The systems may include an in vivo or ex vivo component, wherein a liver function that is compromised in the individual is provided by the disclosed systems comprising liver organoids.

### DESCRIPTION OF THE DRAWINGS

Those of skill in the art will understand that the drawings, described below, are for illustrative purposes only. The drawings are not intended to limit the scope of the present teachings in any way.

FIG. 1 illustrates an exemplary protocol for organoid generation from iPSC and improved cell proliferation using organoid growth medium, and expression of various genes in an organoid formed from an iPSC.

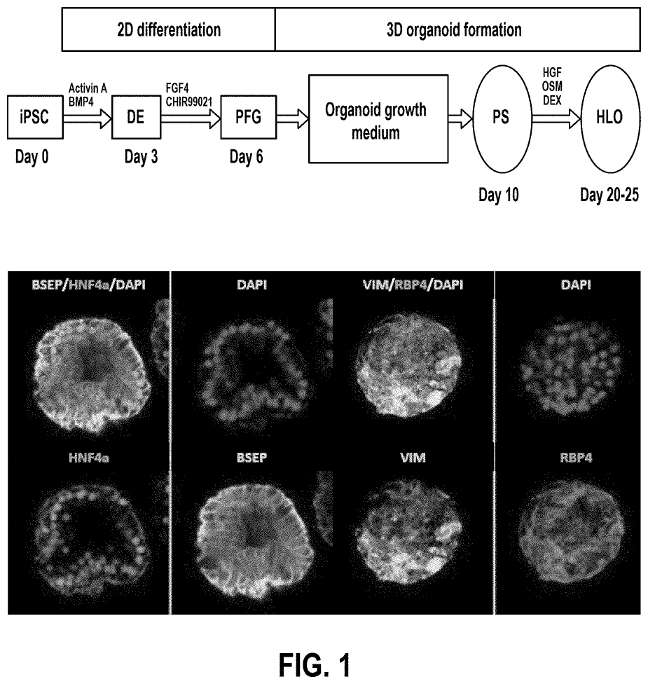

FIG. 2 depicts organoid generation from a primary sample using organoid growth medium.

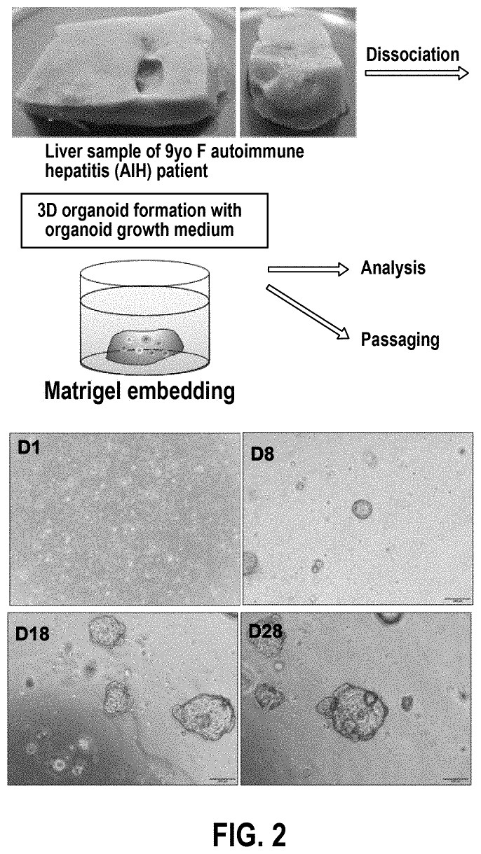

FIG. 3 depicts gene expression analysis of organoids from primary cells.

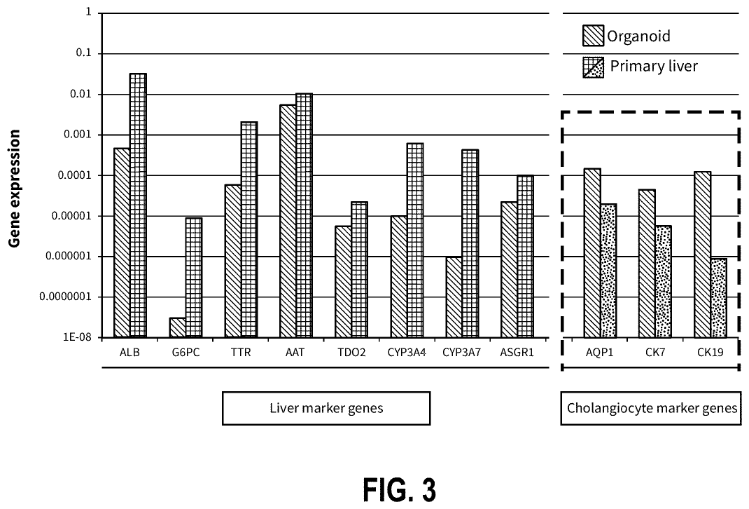

FIG. 4 depicts organoid formation from iPSCs using Matrigel embedding methods and microwell mesh methods.

### DETAILED DESCRIPTION

### Definitions

Unless otherwise noted, terms are to be understood according to conventional usage by those of ordinary skill in the relevant art. In case of conflict, the present document, including definitions, will control. Exemplary methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

As used herein, the term “totipotent stem cells” (also known as omnipotent stem cells) are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

As used herein, the term “pluripotent stem cells (PSCs)” encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

As used herein, the term “induced pluripotent stem cells (iPSCs).” also commonly abbreviated as iPS cells, refers to a type of pluripotent stem cells artificially derived from a normally non-pluripotent cell, such as an adult somatic cell, by inducing a “forced” expression of certain genes, hiPSC refers to human iPSCs. In some embodiments, iPSCs are derived by transfection of certain stem cell-associated genes into non-pluripotent cells, such as adult fibroblasts. Transfection is typically achieved through viral vectors, such as retroviruses. Transfected genes include the master transcriptional regulators Oct-3/4 (Pouf51) and Sox2, although it is suggested that other genes enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include but are not limited to first generation iPSCs, second generation iPSCs in mice, and human induced pluripotent stem cells. In some embodiments, a retroviral system is used to transform human fibroblasts into pluripotent stem cells using four pivotal genes: Oct3/4, Sox2, Klf4, and c-Myc. In alternative embodiments, a lentiviral system is used to transform somatic cells with OCT4, SOX2, NANOG, and LIN28. Genes whose expression are induced in iPSCs include but are not limited to Oct-3/4 (e.g., Pou5fl); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, and LIN28. In some embodiments, non-viral based technologies are employed to generate iPSCs. In some embodiments, an adenovirus can be used to transport the requisite four genes into the DNA of skin and liver cells of mice, resulting in cells identical to embryonic stem cells. Since the adenovirus does not combine any of its own genes with the targeted host, the danger of creating tumors is eliminated. In some embodiments, reprogramming can be accomplished via plasmid without any virus transfection system at all, although at very low efficiencies. In other embodiments, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some embodiments, the expression of pluripotency induction genes can also be increased by treating somatic cells with FGF2 under low oxygen conditions. In some embodiments, exemplary iPS cell lines include but not limited to iPS-DF19-9; iPS-DF19-9; iPS-DF4-3; iPS-DF6-9; iPS(Foreskin); iPS(IMR90); and iPS(IMR90).

As used herein, the term “embryonic stem cells (ESCs),” also commonly abbreviated as ES cells, refers to cells that are pluripotent and derived from the inner cell mass of the blastocyst, an early-stage embryo. For purpose of the present disclosure, the term “ESCs” is used broadly sometimes to encompass the embryonic germ cells as well.

As used herein, the term “precursor cell” encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment.

Artificial liver support systems may treat acute or chronic liver failure by filtering and adsorbing accumulated toxins not cleared by a compromised or nonfunctioning liver, similar to the concept of kidney dialysis systems. Liver support systems may be used to purify the blood by albumin dialysis or plasma separation and filtration, and may be used to remove not only water-soluble substances, but also lipophilic, albumin-bound substances, such as bilirubin, bile acids, metabolites of aromatic amino acids, medium-chain fatty acids and/or cytokines. Artificial livers may remove liver toxins to some degree but lack the ability to carry out the metabolic functions of the liver such as ureagenesis, protein synthesis, gluconeogenesis, enzymatic detoxification, and immune modulation.

Currently available non-biological liver support systems are only designed to perform the role of detoxification, and do not supply synthetic and metabolic functions of the liver. Bio-artificial systems, in contrast, generally contain living cells that can carry out these additional roles. To date, fibroblasts or liver spheroids have been used with such systems (See, e.g., Glorioso et al., J. Hepatol. 2015 August; 63(2): 388-398. doi:10.1016/j.jhep.2015.03.021, disclosing a spheroid reservoir bioartificial liver in a porcine model), however, the function of liver spheroids or primary hepatocytes persist very transiently. Due to the very limited functionality of liver spheroids or primary hepatocytes (typically less than 24 hours), these systems have limited utility. Immortalized hepatocyte cell lines may survive longer but lack critical hepatic functions such as bile acid clearance and ammonium metabolism, and therefore are not ideal for liver support in a patient having such need.

The bioartificial liver systems (“BAL or “Liver Assist Device”) described herein utilize liver organoid technology, and are useful in addressing one or more of the aforementioned needs in the art, in particular, the insufficiency of hepatocytes and/or spheroids in existing bioartificial systems. The disclosed systems and methods employ a liver organoid having a lumenized (or “cavitation”) structure, which are further capable of providing critical hepatic functions to an individual in need thereof. The systems using liver organoids as described herein further address the deficiencies of systems using spheroids or fibroblasts, in that the disclosed liver organoids have persistence of 60 days or more, much longer than the 24 hour viability of fibroblasts and spheroids. These liver organoids are capable of providing bile clearance function and metabolic functions native to the liver, features which are not present in fibroblasts and/or spheroids.

The disclosed liver support systems may include extracorporeal devices categorized as non-biological or bio-artificial liver support systems and can be used to support liver function in an individual in need thereof. The disclosed liver support systems may include the use of supporting or substantially replacing liver function in a patient in need thereof. For example, the disclosed liver support systems may be used to assist liver function in an individual having acute, chronic, and/or end-stage chronic liver failure. The systems are not limited to such individuals, but may further include a use in which the individual does not have a compromised liver function, but is in need of additional support due to a condition in which enhanced liver function would be desirable, whether that be an acute or chronic condition.

In one aspect, a bioartificial liver system (“BAL” or “liver assist device”) is disclosed. The BAL may comprise a liver organoid and a liver organoid support, wherein the liver organoid may be affixed to or contained within said liver organoid support. In one aspect, the bioartificial liver system may provide a liver metabolism function ex vivo, specifically wherein the system is located, at least in part or substantially, outside of the body. In one aspect, the bioartificial liver system may provide a liver metabolism function in vivo specifically wherein the system is located, at least in part or substantially, inside of the body. In one aspect, the liver organoid used in the systems may be characterized in that the liver organoids are able to take up bile unidirectionally from a biological fluid. The biological fluid may be, for example, blood or plasma or combinations thereof. In one aspect, the biological fluid may be blood, and the bioartificial liver system may be implanted in vivo to provide liver function to the individual in which the system is implanted.

It will be understood that in certain circumstance, spheroids may be present in the liver organoid population in the system. In one aspect, however, the ratio of liver organoids to liver spheroids will be in excess of 1:1, such that the majority of a spheroid/organoid population comprises organoids. For example, the bioartificial liver system may comprise a liver organoid/liver spheroid population having less than about 10% liver spheroids, or less than about 5% liver spheroids, or less than about 1% liver spheroids, or which is substantially free of liver spheroids.

The disclosed liver organoids useful in the disclosed systems may be derived from a precursor cell, for example, an embryonic stem cell and/or a pluripotent stem cell. The liver organoid may be characterized by its function. In particular, the liver organoids may have bile clearance function that lasts for at least 30 days, or at least 45 days, or at least 50 days, or at least 60 days, or for over 60 days.

Liver Support System

The support system in which the liver organoids are housed/affixed/contained, may take a variety of different forms, and modification of these features will be within the skill of one of ordinary skill in the art. For example, in one aspect, the support system may comprise a reservoir chamber configured to house a plurality of liver organoids. The support system may be operatively connected to a membrane to allow bi-directional or unidirectional fluid flow. In other aspects, the support system may be used in conjunction with an albumin dialysis system comprising one or more of a blood separation cartridge, a charcoal column, a resin column, and a dialysis membrane. The support system may comprise a chamber or reservoir, in which said liver organoids are free-flowing, wherein said liver organoids may be present at a concentration of from about 10⁶ to about 10¹⁰, or from about 10⁷ to about 10⁹, in a volume of about 500 mL. In one aspect, the liver organoid concentration may be about 1×106⁶ to about 5×106⁶, or about 3×106⁶ in a volume of about 500 mL. The support system may comprise a chamber containing liver organoids in a suspension and may further comprise a mechanism capable of mixing said liver organoid suspension. The liver organoid support system may comprise two fluid paths separated by a permeable medium, wherein communication between the two fluid paths is across the permeable medium, wherein the permeable medium comprises liver organoids, and wherein the liver organoids are in association with said permeable medium. In one aspect, the permeable medium may comprise hollow fibers. In one aspect, the support system may comprise a clinical grade material (i.e., one that is not reactive with biological materials) and may comprise a non-immunogenic material in whole or in part.

In a further aspect, methods of treating an individual having liver failure are disclosed. In certain aspects, the methods may comprise treating an individual in need thereof (whether having compromised biological function or not) with a bioartificial liver system as disclosed herein. The treatment may be carried out for a period of time determined by a medical specialist, but will generally be for a period of time sufficient to provide a favorable therapeutic response in the individual in need thereof. In one aspect, the support system may be provided to an individual in combination with a dialysis system.

Liver Organoids

Liver organoids suitable for use with the disclosed methods and bioartificial liver systems are described herein. In one aspect, the liver organoids useful for the instant disclosure may be characterized by expression of certain genes known to be expressed by liver tissue. For example, the liver organoid may be characterized in that it may express one or more genes selected from PROX1, RBP4, CYP2C9, CYP3A4, ABCC11. CFH, C3, C5, ALB, FBG, MRP2, ALCAM, CD68, CD34, CD31. In further aspects, the liver organoids may express certain proteins such as alpha-fetoprotein (AFP), albumin (ALB), retinol binding protein (RBP4), cytokeratin 19 (CK19), hepatocyte nuclear factor 6 (HNF6), and cytochrome P450 3A4 (CYP3A4), HNF4a, E-cadherin, DAPI, and Epcam. Such expression may occur, for example, at day 40 to day 50 of the developing liver organoid. The expression level of the aforementioned genes/proteins may be similar to that observed in human liver cells, for example, that of an adult liver cell. In one aspect, the liver organoid may be characterized by having total hepatic protein expression of at least 10,000 ng/mL 1×e6 cells/24 hours.

The liver organoids useful for the instant disclosure may also be characterized by their structure. The liver organoid may comprise a luminal structure further containing internalized microvilli and mesenchymal cells. The luminal structure of the liver organoid may be surrounded by polarized hepatocytes and a basement membrane. The liver organoid may comprise functional stellate cells and functional Kupffer cells. In one aspect, the liver organoid may comprise cells comprising a drug metabolism cytochrome variant, such as, for example, a CY2C9*2 variant. The liver organoid may comprise a vasculature, such as that described in US 20160177270. In one aspect, the liver organoid may be characterized in that the liver organoid does not comprise inflammatory cells, for example T-cells or other inflammatory secreted proteins.

The liver organoids useful for the instant disclosure may also be characterized by their function. For example, the liver organoid may be characterized by having one or more of the following: bile production capacity, bile transport activity, Complement factor H expression of at least 50 ng/mL/1×e6 cells/24 hr, Complement factor B of at least 40 ng/mL/1×e6 cells/24 hr, C3 expression of at least 1000 ng/mL/1×e6 cells/24 hr; C4 expression of at least 1000 ng/mL/1×e6 cells/24 hr, fibrinogen production of at least 1,000 ng/mL/1×e6 cells/24 hr and albumin production of at least 1.000 ng/mL/1×e6 cells/24 hr. In one aspect, the liver organoid may be capable of taking up bile unidirectionally from a biological fluid, preferably wherein said biological fluid is selected from blood or plasma. While 3D aggregated liver cells have been reported, the disclosed compositions have very high functional activity such as albumin production (up to a 10-fold increase compared with conventional highest standard models using iPSC-derived hepatocytes), and allow for improved oxygen and/or nutrition supply due to the internal luminal structure, which allows for much longer culture (in some instances, at least over 60 days). In one aspect, the liver organoid used herein has bile clearance function for at least 30 days, or at least 45 days, or at least 50 days, or at least 60 days, or for over 60 days.

The disclosed liver organoids may be derived from a precursor cell, preferably a pluripotent stem cell, using methods known in the art. Precursor cells include but are not limited to any cell that can be used in methods described herein, through which one or more precursor cells acquire the ability to renew itself or differentiate into one or more specialized cell types. In some aspects, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. In addition, mixtures of precursor cells from different cell strains, mixtures of normal and genetically modified cells, or mixtures of cells from two or more species or tissue sources may be used for treating patients with liver failure. Cells can be genetically modified using, without limitation, spontaneous, chemical, or viral methods, including CRISPR methods known in the art. Genetically modified cells can exhibit characteristics such as immortality, reduced allogenicity, or differentiated hepatocyte function.

In one aspect, the liver organoids may be cultured in a basement membrane matrix for a period of time sufficient to enhance the ability of said liver organoid to take up bile acid unidirectionally. The cultured liver organoids may then be transplanted to the bioartificial liver system. In manufacturing the disclosed liver organoids for the disclosed system, it may be advantageous to culture the disclosed liver organoids a basement membrane matrix, such as Matrigel™ until the liver organoid is capable of taking up bile acid unidirectionally.

Methods of Making Liver Organoids

Methods of manufacturing liver organoids have been described by Applicant. See. e.g., WO2018085615A1 to Takebe, “Liver organoid compositions and methods of making and using same.” In one aspect, a method of inducing formation of a liver organoid from iPSC cells is disclosed. The method may comprise the steps of a) contacting definitive endoderm (DE) derived from iPSC cells with a FGF pathway activator and a GSK3 inhibitor (which acts as a Wnt signaling pathway activator), for a period of time sufficient to form posterior foregut spheroids, preferably for a period of time of from about 1 day to about 3 days and b) incubating the resulting posterior foregut spheroids of step a in the presence of retinoic acid (RA) for a period of time sufficient to form a liver organoid, preferably for a period of time of from about 1 to about 5 days, preferably about 4 days. The liver organoid may be made according to methods known in the art, for example, using the methods disclosed in PCT/US2018/027585. In particular, in one aspect, the iPSC derived foregut may be subjected to freezing conditions, for example, about −80C, prior to organoid formation. In one aspect, the iPSC derived foregut may be subjected to freezing conditions as single cells (in contrast to a cell cluster).

Organoid formation efficiency has been demonstrated by applicant to be improved when formed from frozen single cells. See, e.g., PCT/US2018/027585. Such method may be used with the liver support systems disclosed herein. For example, Applicant has invented a freezing method of obtaining liver organoids from an iPSC derived foregut cell. As a result of freezing and thawing, the survival rate of single cell isolated cells was significantly higher than a cluster condition cell. Specifically, freezing an iPSC derived foregut cell allows for more efficient organoid formation from isolated single cells. Such method may be used with the present disclosure to improve manufacture of the liver organoids and/or systems described herein.

Fibroblast growth factors (FGFs) are a family of growth factors involved in angiogenesis, wound healing, and embryonic development. The FGFs are heparin-binding proteins and interactions with cell-surface associated heparan sulfate proteoglycans have been shown to be essential for FGF signal transduction. Suitable FGF pathway activators will be readily understood by one of ordinary skill in the art. Exemplary FGF pathway activators include, but are not limited to: one or more molecules selected from the group consisting of FGF1, FGF2, FGF3, FGF4, FGF10, FGF11, FGF12, FGF13, FGF14, FGF15, FGF16, FGF17. FGF18, FGF19, FGF20, FGF21, FGF22, and FGF23. In some embodiments, siRNA and/or shRNA targeting cellular constituents associated with the FGF signaling pathway may be used to activate these pathways.

In some embodiments, DE culture is treated with the one or more molecules of the FGF signaling pathway described herein at a concentration of 10 ng/ml or higher; 20 ng/ml or higher; 50 ng/ml or higher; 75 ng/ml or higher; 100 ng/ml or higher; 120 ng/ml or higher, 150 ng/ml or higher; 200 ng/ml or higher; 500 ng/ml or higher; 1,000 ng/ml or higher; 1,200 ng/ml or higher; 1,500 ng/ml or higher; 2,000 ng/ml or higher, 5,000 ng/ml or higher; 7,000 ng/ml or higher; 10,000 ng/ml or higher, or 15,000 ng/ml or higher. In some embodiments, concentration of signaling molecule is maintained at a constant throughout the treatment. In other embodiments, concentration of the molecules of a signaling pathway is varied during the course of the treatment. In some embodiments, a signaling molecule in accordance with the present invention is suspended in media comprising DMEM and fetal bovine serine (FBS). The FBS can be at a concentration of 2% and more; 5% and more; 10% or more; 15% or more; 20% or more; 30% or more; or 50% or more. One of skill in the art would understand that the regiment described herein is applicable to any known molecules of the signaling pathways described herein, alone or in combination, including but not limited to any molecules in the FGF signaling pathway.

Suitable GSK3 inhibitors will be readily understood by one of ordinary skill in the art. Exemplary GSK3 inhibitors include, but are not limited to: Chiron/CHIR99021, for example, which inhibits GSK3β. One of ordinary skill in the art will recognize GSK3 inhibitors suitable for carrying out the disclosed methods. The GSK3 inhibitor may be administered in an amount of from about 1 μM to about 100 μM, or from about 2 μM to about 50 μM, or from about 3 μM to about 25 μM. One of ordinary skill in the art will readily appreciate the appropriate amount and duration. Wnt signaling pathway activators may be substituted for the disclosed GSK3 inhibitors. Such substitution is understood in the art, and may be readily determined by routine experimentation by one of ordinary skill in the art.

In one aspect, the stem cells may be mammalian, or human, iPSCs. As described above, the foregut spheroids may be embedded in a basement membrane matrix, such as, for example, the commercially available basement membrane matrix sold under the tradename Matrigel.

Bioartificial Liver Systems

In one aspect, the bioartificial liver system may provide a liver metabolism function to an individual in need thereof, wherein said liver metabolism function is provided using a bioartificial liver system, wherein the bioartificial liver system is at least in part located ex vivo to the individual in need thereof. In one aspect, the bioartificial liver system may provide a liver metabolism function to an individual in need thereof, wherein said bioartificial liver system is located at least in part in vivo to the individual in need thereof. Such function may be carried out, for example, by incorporation of the described liver organoids in conjunction with a liver assist device. The liver assist device may take the form of any device known in the art. For example, the bioartificial liver system (liver assist device) may take a variety of different forms, for example, that described in U.S. Pat. No. 9,650,609. US20120009086, WO1992007615A1, US20030228685A1, wherein the biological function is provided by, at least in part, a liver organoid composition. For example, the liver assist device may comprise a bioreactor that includes a liver organoid composition as described herein, the liver organoids being located within a cell compartment and/or cell reservoir, optionally supplemented with additional cell types and/or spheroids. Hepatocytes, or other cells present in the liver, also can be included in the system in addition to the liver organoids, such as endothelial cells, Ito cells, Kupffer cells, and fibroblasts. A co-culture of hepatocytes with one or more of these or other types of cells also can be used in a bioartificial liver system/liver assist device.

Further examples of systems that may be used in conjunction with the described liver organoid systems include the Molecular Adsorbent Recirculating System, or “MARS” system (Gambro Americas, Lakewood, Colo.), Plasma Separation Adsorption and Dialysis system (Prometheus. Fresenius Medical Care. Bad Homburg, Germany), single-pass albumin dialysis (SPAD), and selective plasma filtration therapy (SEPET). Other systems include single pass albumin dialysis (SPAD), Therapeutic Plasma Exchange (TPE), which involves extra coroporeal separation and removal of patient plasma from blood ant the return of blood cells with a replacement fluid to the patient, Extracorporeal Liver Assist Device (ELAD), the AMC-BAL device, the Spheroid Reservoir Bioartifical Device (SRBAL), the Hepa Wash, Li-ALS, and the University College London-Liver Dialysis Deice (UCL-LDD).

In one aspect, the system used in conjunction with the disclosed liver organoids is the MARS device. MARS consists of a double-circuit system, one circuit with a haemodiafilter and the other with albumin as the acceptor for albumin-bound toxins, which can remove both water soluble and lipophilic molecules. The fractionated plasma separation and adsorption system (Prometheus) is also a double-circuit system and consists of a blood circuit and a plasma circuit separated by an albumin-permeable polysulfone membrane. The plasma fraction passes into the absorbents that comprise an anion exchanger and a neutral resin in which the albumin-bound toxins are directly purified, while the blood circuit is exposed to high-flux dialysis to remove water soluble toxins.

In one aspect, liver organoids may be placed in a 3D network of hollow fibers designed for plasma perfusion. The liver organoids may be used with, for example, the HepatAssist™ system (Alliqua Inc., Langhorne, Pa., USA), the most widely used BAL device, using liver organoids in conjunction with a dialysis cartridge such that the patients' plasma encounters the liver organoids across a micro-porous membrane.

In one aspect, the liver organoids may be used with the Extracorporeal Liver Assist Device (ELAD®; Vital Therapies Inc., San Diego, Calif., USA), in which the liver organoids may provide liver enzymatic activities. Other designs that may be used with the liver organoids described herein include the Amsterdam Medical Center bioartifical liver, “AMC-BAL” (Center, Amsterdam, Netherlands) and the MELS (Modular Extracorporeal Liver Support System, Charité, Berlin, Germany).

In one aspect, the liver organoids may be used with the Bioartificial Liver Support System (BLSS™), by Excorp Meical (Minneapolis, Minn.), in which the primary porcine hepatocytes in the single hollow cartridge may be substituted with (in whole or in part) the liver organoids as described herein.

The aforementioned systems are described in McKenzie et al, “Artificial and Bioartificial Liver Support,” Seminars in Liver Disease, Vol. 28, No. 2 (2008), which is incorporated herein in its entirety by reference.

In one aspect, the liver organoids as described herein may be used with the device described in U.S. Pat. No. 9,650,609 to Nyberg, filed Jun. 17, 2014; US2012/0009086 to Nyberg, filed Mar. 12, 2010, PCT/US91/07952 to Shatford et al, published May 14, 1992, each of which is incorporated herein in its entirety by reference. For example, where fibroblasts or other cell types that assist in liver function are described, liver organoids as described herein may be substituted.

In general, the disclosed systems may employ a liver organoid (which includes a “plurality of organoids”) in conjunction with a liver organoid support wherein the combination is utilized in a bioartificial liver support system to assist liver function. By “liver organoid support,” it is meant any substrate or container that may affix, contain, or in some manner orient liver organoids in a manner that allows the liver organoid(s) to contact the biological fluid sufficient to assist liver function, whether in the form of a cartridge or hollow fiber as described in any of the systems referenced above, or in an alternative form that serves as a substrate for providing liver organoids as part of the liver support system. Examples of liver organoid supports include, but are not limited to, hollow fibers such as those described in US 2011/0218512, cartridges, and may take any shape desired, such as a cylindrical shape, a substantially planar shape, or any modification thereof. In one aspect, the liver organoid support maintains the liver organoid(s) in a desired configuration. The liver organoid support may, in one aspect, comprise a reservoir chamber configured to house liver organoids. The liver organoid support may be, in certain aspects, operatively connected to a membrane to allow bi-directional fluid flow, and/or may be operatively connected to a membrane to allow unidirectional fluid flow.

In one aspect, the bioartificial liver system described herein may be used in conjunction with an albumin dialysis system comprising one or more of a blood separation cartridge, a charcoal column, a resin column, and a dialysis membrane.

In one aspect, the liver organoid support may comprise a chamber or reservoir, in which the liver organoids may be capable of free-flowing or movement within the chamber. The liver organoid support may comprise a chamber containing the liver organoids in a suspension and may further comprise a mechanism capable of mixing said liver organoid suspension.

In one aspect, the liver organoid support may comprise two fluid paths separated by a permeable medium, wherein communication between the two fluid paths is across the permeable medium, wherein the permeable medium may comprise liver organoids, and wherein the liver organoids may be in association with the permeable medium. In one aspect, the permeable medium may comprise hollow fibers such as those described in US20150273127A1, to Flieg et al, filed Nov. 26, 2012. In one aspect, the liver organoid support may be of a clinical grade and non-immunogenic.

In one aspect, the bioartificial liver system may comprise a liver organoid and a biomaterial. The biomaterial may be a hydrogel. In one aspect, the biomaterial may be selected from collagen, alginate, fibrin, gelatin, alginate-fibrin hydrogels, collagen-fibrinogen hydrogels, collagen-alginate hydrogels, fibrogen-alginate hydrogels, Collagen-Alginate-Fibrin (CAF) hydrogels, and combinations thereof. See, e.g., Montalbano et al, Synthesis of bioinspired collagen/alginate/fibrin-based hydrogels for soft tissue engineering, Materials Science and Engineering, Volume 91, 1 Oct. 2018. Pages 236-246 for exemplary materials that may be used with the disclosed systems.

Organoid Growth Medium

In one aspect, the medium used for growth of liver organoids, and/or for use within the liver device systems may comprise advanced DMEM/F12 comprising the following: B27 (for example, about 1-5%, or about 2% B27). N2 supplement (for example, about 0.1 to about 5%, or about 1% N2 supplement), Gultamaz (for example, about 0.1% to about 10%, or about 1% Gultamaz), HEPES (for example, about 0.1% to about 10%, or about 1% HEPES), ascorbic acid (for example, about 5 to about 100 ug/mL ascorbic acid, or about 50 ug/mL ascorbic acid), FGF2 (for example, about 1 to about 100 ng/mL, or about 5 ng/mL FGF2), VEGF (for example, about 1 mg/mL to about 100 mg/mL, or about 10 mg/mL VEGF), EFG (for example, about 1 ng/mL to about 100 nm/mL, or about 20 ng/mL EGF), CHIR99021 or other FGF signaling pathway activator (for example, about 1-50 μM CHIR99021, or about 3 μM CHIR99021), and A83-01 (for example, about 0.01 to about 1 μM, or about 0.5 μM A83-01). In one aspect, the organoid growth medium may each of the above listed components in an amount of from about one half of the above listed amount to about twice the above listed amount, or from about one quarter of the above listed amount to about three times the above listed amount.

Method of Treatment

In one aspect, disclosed is a method of treating an individual having compromised liver function for example, liver failure, comprising the step of providing a bioartificial liver system (“BAL” or “liver assist device,” used interchangeably herein) as described herein to an individual in need thereof, optionally, further in combination with a dialysis system. The term “liver failure” refers to the inability of the liver to perform its normal synthetic and metabolic function as part of normal physiology. Liver failure thus leads, for example, to an insufficient detoxification of albumin, which is followed by an exhaustion of the binding capacity of albumin and an enrichment of the otherwise albumin-bound toxins, e.g. of unconjugated bilirubin. Treatment is indicated, for example, at a bilirubin concentration of >10 mg/dL. However, there are liver disorders where a liver dialysis treatment is indicated, but which is not characterized by increased bilirubin levels. Disorders which are associated with the expression “liver failure” as used may include, but are not limited to, hepatorenal syndrome, decompensated chronic liver disease, acute liver failure, graft dysfunction after liver transplantation, liver failure after liver surgery, secondary liver failure, multi organ failure, exogenous intoxication or intractable pruritus in cholestasis etc.

Examples

Liver Organoid Preparation Methods

Differentiation into liver organoids. Three methods may be used to differentiate the DE into liver organoids: The “Matrigel Drop Method,” the “Matrigel Sandwich Method,” and the Matrigel-Free Method,” each of which is described below.

Matrigel Drop Method: On Day 7-8, definitive endoderm organoids with plated cells were gently pipetted to delaminate from dishes. Isolated spheroids were centrifuged at 800 rpm for 3 minutes and, after removing supernatant, embedded in 100% matrigel drop on the dishes. The plates were placed at 37° C. in an atmosphere of 5% CO₂/95% air for 5-15 min. After the Matrigel was solidified. Advanced DMEM/F12 was added with B27, N2 and Retinoic acid (RA; Sigma, St. Louis, Mo.) 2 μM for 1-5 days. The media was replaced every other day. After RA treatment, organoids embedded in Matrigel drop were cultured in Hepatocyte culture medium (HCM Lonza, Walkersville, Md.) with 10 ng/mL hepatocyte growth factor (HGF; PeproTech. Rocky Hill. N.J.), 0.1 μM Dexamethasone (Dex; Sigma) and 20 ng/mL Oncostatin M (OSM; R&D Systems). Cultures for cell differentiation were maintained at 37° C. in an atmosphere of 5% CO₂/95% air and the medium was replaced every 3 days. Around Day 20-30, organoids embedded in Matrigel drop were isolated by scratching and gentle pipetting for any analyses.

Example using Matrigel Drop Method

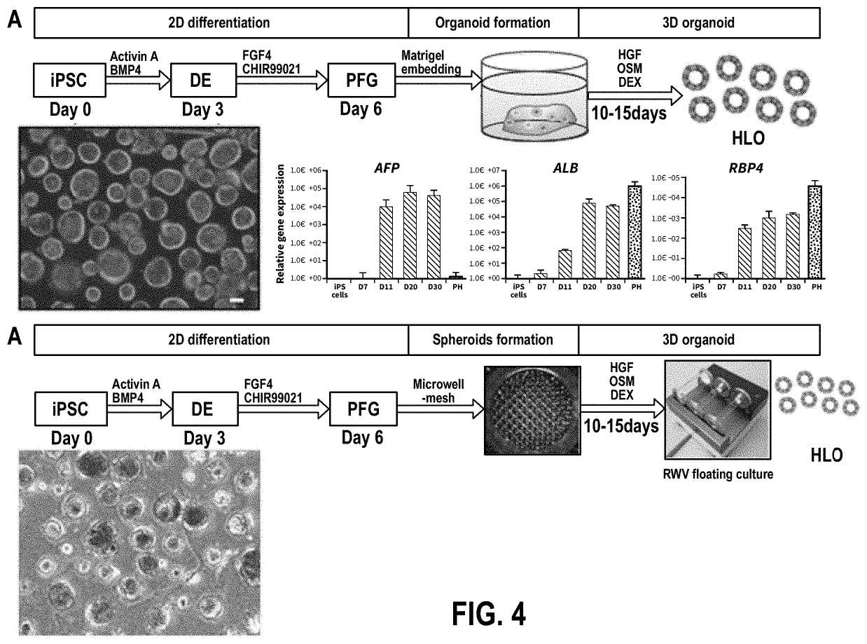

A large number of functional HLOs may be generated using Matrigel drops. Human iPSC lines may be differentiated into definitive endoderm (DE), and then posterior foregut (PFG). Next, the differentiated cells may be embedded into 50-70 ul Matrigel drops (3D). After 2 weeks in the maturation stage using hepatocyte culture medium (HCM), each Matrigel drop will generate around a hundred HLOs. These HLOs highly express ALB, AFP, and RBP4 (FIG. 4. Panel A). To further scale up the production of HLOs, microwell-mesh plates may be used to generate uniformly sized spheroids. These microwell-mesh plates contain 500 um in diameter with 100 um in depth microwells which can efficiently form a large number of spheroids. Spheroids may then be transferred into a rotation wall vessel bioreactor (RWV) with HCM. FIG. 4, Panel B shows organoids formation.

An exemplary protocol is as follows:

Seed PFG single cells in to EZSPHERE™ six well microwell-mesh plates. (This may be substituted by other microwell plates such as Aggrewell™, Elplasia or the like.) Seed 2×10\hat{ }6 cells/well with Advanced DMEM+RA+Rock inhibitor. Gently spin the plate at 160 g for 1 min. Put the plate into an incubator at 37 C, 5% CO2 overnight.

On the second day, spheres should be observed. Gently pipette the medium up and down, then collect spheres in to a 15 ml tube. Each well in the 6 well plate can generate approximately 2000 spheres. For the whole plate, about 12000 spheres will be generated.

Allow the spheres and medium mixture sit on the bench for 5 min until the spheres are all settle down to the bottom. Aspirate all the medium. Change the medium and mix in with 10% of Matrigel. Transfer all the spheres and the medium into the rotation wall vessel bioreactor (RWV) (RWV or equivalent system is commercially available from multiple vendors such as JTEC corporation, Synthecon, SATAKE, and ABLE). Incubate at 37 C with 5% CO2. Change medium every 3 days. Liver organoids will be formed after 14 days.

Matrigel Sandwich Method: On Day 7-8, definitive endoderm organoids with plated cells were gently pipetted to delaminate from dishes. Isolated spheroids were centrifuged at 800 rpm for 3 minutes, and after removing supernatant, they were mixed with 100% Matrigel. At the same time, hepatocyte culture medium with all supplements was mixed with the same volume of 100% Matrigel. HCM and Matrigel mix was plated to the bottom of dish to make a thick coating on the plate (0.3-0.5 cm), and placed at 37° C. in an atmosphere of 5% CO₂/95% air for 15-30 min. After the Matrigel was solidified, spheroids mixed with Matrigel was seeded on Matrigel thick coated plated. The plate was placed at 37° C. in an atmosphere of 5% CO₂/95% air for 5 min. Advanced DMEM/F12 was added with B27, N2 and Retinoic acid (RA; Sigma, St. Louis, Mo.) 2 μM for 1-5 days. The media was replaced every other day. After RA treatment, organoids embedded in Matrigel drop were cultured in Hepatocyte culture medium (HCM Lonza, Walkersville, Md.) with 10 ng/mL hepatocyte growth factor (HGF; PeproTech, Rocky Hill, N.J.), 0.1 μM Dexamethasone (Dex; Sigma) and 20 ng/mL Oncostatin M (OSM; R&D Systems). Cultures for cell differentiation were maintained at 37° C. in an atmosphere of 5% CO₂/95% air and the medium was replaced every 3 days. Around Day 20-30, organoids embedded in Matrigel drop were isolated by scratching and gentle pipetting for any analyses.

Matrigel-Free Method: On Day 7-8, definitive endoderm organoids with plated cells were continued planar culture in Advanced DMEM/F12 (Thermo Fisher Scientific Inc.) with B27 (Life Technologies) and N2 (Gibco, Rockville, Md.) Retinoic acid (RA; Sigma, St. Louis, Mo.) 2 μM for 4 days. The media was replaced every other day. After the 4 days planar culture, the organoids begin to bud, whereas 2D cells differentiate into hepatocytes. Both organoids and hepatocytes can be maintained for over 60 days under Hepatocyte culture medium (HCM Lonza, Walkersville, Md.) with 10 ng/mL hepatocyte growth factor (HGF; PeproTech, Rocky Hill, N.J.), 0.1 μM Dexamethasone (Dex: Sigma) and 20 ng/mL Oncostatin M (OSM; R&D Systems) for 10 days. For organoid assays, floating organoids can be collected in Ultra-Low attachment multiwell plates 6 well plate and used for subsequent assays whenever appropriate. Cultures for cell differentiation were maintained at 37° C. in an atmosphere of 5% CO₂/95% air and the medium was replaced every 3 days.

### Example: Generation and Characterization of Polarized Liver Organoids

BMP and Activin A are used to promote differentiation into definitive endoderm as previously described (D'Amour et al., 2005). Any methods for producing definitive endoderm from pluripotent cells (e.g., iPSCs or ESCs) are applicable to the methods described herein. Exemplary methods are disclosed in, for example “Methods and systems for converting precursor cells into intestinal tissues through directed differentiation,” U.S. Pat. No. 9,719,068B2 to Wells et al., and “Methods and systems for converting precursor cells into gastric tissues through directed differentiation.” US20170240866A1, to Wells et al. In some embodiments, pluripotent cells are derived from a morula. In some embodiments, pluripotent stem cells are stem cells. Stem cells used in these methods can include, but are not limited to, embryonic stem cells. Embryonic stem cells can be derived from the embryonic inner cell mass or from the embryonic gonadal ridges. Embryonic stem cells or germ cells can originate from a variety of animal species including, but not limited to, various mammalian species including humans. In some embodiments, human embryonic stem cells are used to produce definitive endoderm. In some embodiments, human embryonic germ cells are used to produce definitive endoderm. In some embodiments, iPSCs are used to produce definitive endoderm. Additional methods for obtaining or creating DE cells that can be used in the present invention include but are not limited to those described in U.S. Pat. No. 7,510,876 to D'Amour et al.; U.S. Pat. No. 7,326,572 to Fisk et al.; Kubol et al., 2004, “Development of definitive endoderm from embryonic stem cells in culture,” Development 131:1651-1662: D'Amour et al., 2005, “Efficient differentiation of human embryonic stem cells to definitive endoderm,” Nature Biotechnology 23:1534-1541; and Ang et al., 1993, “The formation and maintenance of the definitive endoderm lineage in the mouse: involvement of HNF3/forkhead proteins,” Development 119:1301-1315, all of which are incorporated in their entirety by reference herein.

FGF4, and a GSK3 inhibitor (CHIR99021) are used to induce foregut spheroids and budded spheroids. Organoids are embedded in Matrigel after delamination with mesenchymal cells plated on the dish by gentle pipetting. To generate polarized organoids suited for bile transport modeling, organoids are treated with RA. To optimize the organoid generation method, RA treatment may be varied. Duration of RA of 4 days based on the level of albumin secretion is typically used. Around 10 days after RA treatment, over 300 organoids covered with epithelial cells can be successfully generated, and the ratio of organoids with lumenized structure is about 70%. The resulting organoids are albumin positive in epithelial cells, and Type IV collagen is localized to the outer surface and ZO-1 (zonula occludens) stained the intraluminal lining.

Cells in the resulting organoids have a significant increase in expression of hepatic marker genes such as alpha-fetoprotein (AFP), albumin (ALB), retinol binding protein 4 (RBP4), Cytokeratin 19 (CK19), hepatocyte nuclear factor 6 (HNF6) which controls cholangiocyte differentiation, and Cytochrome P450 3A4 (CYP3A4) during differentiation. Approximately 30% of cells are non-parenchymal cells which were identified by stromal cell markers (Unpublished observation), making the organoids more similar to in vivo liver tissue than primary hepatocytes.

Example Liver Support System using Liver Organoids

Organoid (HLO) can be grown indefinitely in a 3D rotating bioreactor (JTEC corporation (but not limited to JTEC system)) in special medium (attached formulation). Finally, about 3×10⁹ human hepatocyte equivalent HLO are harvested from the reactor and used for the HLO-BAL treatment.

BAL System

The BAL system consists of a cell circuit and a blood circuit. The components of BAL system include three roller pumps, a heparin pump, a plasma filter (Sorin Group Italia, Mirandola. Italy), a plasma component separator (Kawasumi Laboratories Inc. Tokyo, Japan), and a multi-layer flat-plate bioreactor with polycarbonate scaffolds. As previously reported (Han et al., 2012; Shi et al., 2011), the membrane within the plasma component separator is made from ethylene vinyl alcohol copolymer resin with excellent biocompatibility. The pore size of membranes is 10 nm (˜200 KD). The membrane molecular weight cutoff showed high performance in decrease IgG, IgM, deposition of which may reduce the severe xenoreactive antibody response and effectively remove target substance as well (Han et al., 2012; Shi et al., 2011).

As reported (Shi et al., 2012), the multi-layer bioreactor consists of a hollow column stent, and a stack of 65-layer round flat plates, all of which are made of polycarbonate. The channel height between neighboring plates is maintained at 0.5 mm with the spacers attached to the bottom of each plate. The HLO are implanted into the bioreactor through the eyelets by a peristaltic pump. The height of the bioreactor is about 10 cm, and the effective volume is 480 ml. Freshly harvested hiHep cells were transported in HMM. The whole bioreactor is incubated at 37° C. and 5% CO2 overnight until the cells are adhered to the surface of plates and ready for the BAL treatment.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A bioartificial liver system (“BAL” or “liver assist device”) comprising a liver organoid and a liver organoid support, wherein said liver organoid is affixed to or contained within said liver organoid support.

2. The bioartificial liver system of claim 1, wherein said bioartificial liver system provides a liver metabolism function ex vivo.

3. The bioartificial liver system of claim 1, wherein said bioartificial liver system provides a liver metabolism function in vivo.

4. The bioartificial liver system of claim 1, wherein said liver organoid takes up bile unidirectionally from a biological fluid, preferably wherein said biological fluid is one or both of blood or plasma.

5. The bioartificial liver system of claim 1, wherein said biological fluid is blood, and wherein said bioartificial liver system is implanted in vivo.

6. The bioartificial liver system of claim 1, wherein said bioartificial liver system comprises a liver organoid population having less than about 10% liver spheroids, or less than about 5% liver spheroids, or less than about 1% liver spheroids, or which is substantially free of liver spheroids.

7. The bioartificial liver system of claim 1, wherein said liver organoid comprises a lumen.

8. The bioartificial liver system of claim 1, wherein said liver organoid is derived from a precursor cell, preferably one or both of an embryonic stem cell and pluripotent stem cell.

9. The bioartificial liver system of claim 1, wherein said liver organoid has bile clearance function for at least 30 days, or at least 45 days, or at least 50 days, or at least 60 days, or for over 60 days.

10. The bioartificial liver system of claim 1, wherein said liver organoids are cultured in a basement membrane matrix until said liver organoid is capable of taking up bile acid unidirectionally.

11. The bioartificial liver system of claim 1, wherein said liver organoid support is capable of maintaining said liver organoids in a desired configuration.

12. The bioartificial liver system of claim 1, wherein said liver organoid support comprises a reservoir chamber configured to house liver organoids.

13. The bioartificial liver system of claim 1, wherein said liver organoid support is operatively connected to a membrane to allow bi-directional fluid flow.

14. The bioartificial liver system of claim 1, wherein said liver organoid support is operatively connected to a membrane to allow unidirectional fluid flow.

15. The bioartificial liver system of claim 1, wherein said bioartificial liver support is used in conjunction with an albumin dialysis system comprising one or more of a blood separation cartridge, a charcoal column, a resin column, and a dialysis membrane.

16. The bioartificial liver system of claim 1, wherein said liver organoid support comprises a chamber or reservoir, in which said liver organoids are free-flowing, preferably wherein said liver organoids are in a concentration of from about 10⁶ to about 10¹⁰, or from about 10⁷ to about 10⁹, in a volume of about 500 mL.

17. The bioartificial liver system of claim 1, wherein said liver organoid support comprises a chamber containing said liver organoids in a suspension, further comprising a mechanism capable of mixing said liver organoid suspension.

18. The bioartificial liver system of claim 1, wherein said liver organoid support comprises two fluid paths separated by a permeable medium, wherein communication between the two fluid paths is across said permeable medium, wherein said permeable medium comprises liver organoids, wherein said liver organoids are in association with said permeable medium.

19. The bioartificial liver system of claim 1, wherein said permeable medium comprise hollow fibers.

20. The bioartificial liver system of claim 1, wherein said liver organoid support is of a clinical grade and wherein said liver organoid support is non-immunogenic.

21. The bioartificial liver system of claim 1, wherein said bioartificial liver system is provided to an individual in need thereof, further in combination with a dialysis system.

22. A method of treating an individual having liver failure comprising treating said individual with a bioartificial liver system of claim 1.
